SEQUENCE LISTING (113 sequences)

SEQ ID NO: 1 - protein, 4 aa, Human papillomavirus
GPAG

SEQ ID NO: 2 - protein, 4 aa, Human papillomavirus
LQPE

SEQ ID NO: 3 - protein, 4 aa, Human papillomavirus
LEDL

SEQ ID NO: 4 - protein, 4 aa, Human papillomavirus
DTPT

SEQ ID NO: 5 - protein, 4 aa, Human papillomavirus
QSTH

SEQ ID NO: 6 - protein, 4 aa, Human papillomavirus
ETTD

SEQ ID NO: 7 - protein, 4 aa, Human papillomavirus
TTDL

SEQ ID NO: 8 - protein, 4 aa, Human papillomavirus
LNDS

SEQ ID NO: 9 - protein, 4 aa, Human papillomavirus
EEED

SEQ ID NO: 10 - protein, 4 aa, Human papillomavirus
STLR

SEQ ID NO: 11 - protein, 4 aa, Human papillomavirus
PTLH

SEQ ID NO: 12 - protein, 4 aa, Human papillomavirus
TLRL

SEQ ID NO: 13 - protein, 4 aa, Human papillomavirus
EEDE

SEQ ID NO: 14 - protein, 4 aa, Human papillomavirus
DEID

SEQ ID NO: 15 - protein, 4 aa, Human papillomavirus
IRTL

SEQ ID NO: 16 - protein, 4 aa, Human papillomavirus
DSTL

SEQ ID NO: 17 - protein, 8 aa, Human papillomavirus
IDGPAGQA

SEQ ID NO: 18 - protein, 8 aa, Human papillomavirus
EDEIDGPA

SEQ ID NO: 19 - protein, 9 aa, Human papillomavirus
QLNDSSEEE

SEQ ID NO: 20 - protein, 9 aa, Human papillomavirus
EIDGPAGQA

SEQ ID NO: 21 - protein, 10 aa, Human papillomavirus
QLNDSSEEED

SEQ ID NO: 22 - protein, 10 aa, Human papillomavirus
NDSSEEEDEI

SEQ ID NO: 23 - protein, 9 aa, Human papillomavirus
EEDEIDGPA

SEQ ID NO: 24 - protein, 10 aa, Human papillomavirus
DEIDGPAGQA

SEQ ID NO: 25 - protein, 10 aa, Human papillomavirus
EEEDEIDGPA

SEQ ID NO: 26 - protein, 9 aa, Human papillomavirus
SEEEDEIDG

SEQ ID NO: 27 - protein, 9 aa, Human papillomavirus
SSEEEDEID

SEQ ID NO: 28 - protein, 10 aa, Human papillomavirus
SSEEEDEIDG

SEQ ID NO: 29 - protein, 10 aa, Human papillomavirus
EIDGPAGQAE

SEQ ID NO: 30 - protein, 9 aa, Human papillomavirus
EEEDEIDGP

SEQ ID NO: 31 - protein, 10 aa, Human papillomavirus
EEDEIDGPAG

SEQ ID NO: 32 - protein, 9 aa, Human papillomavirus
EDEIDGPAG

SEQ ID NO: 33 - protein, 9 aa, Human papillomavirus
DSSEEEDEI

SEQ ID NO: 34 - protein, 9 aa, Human papillomavirus
EQLNDSSEE

SEQ ID NO: 35 - protein, 8 aa, Human papillomavirus
DGPAGQAE

SEQ ID NO: 36 - protein, 8 aa, Human papillomavirus
SSEEEDEI

SEQ ID NO: 37 - protein, 9 aa, Human papillomavirus
DEIDGPAGQ

SEQ ID NO: 38 - protein, 8 aa, Human papillomavirus
EIDGPAGQ

SEQ ID NO: 39 - protein, 15 aa, Human papillomavirus
SEEEDEIDGPAGQAE

SEQ ID NO: 40 - protein, 15 aa, Human papillomavirus
QLNDSSEEEDEIDGP

SEQ ID NO: 41 - protein, 15 aa, Human papillomavirus
DSSEEEDEIDGPAGQ

SEQ ID NO: 42 - protein, 15 aa, Human papillomavirus
SSEEEDEIDGPAGQA

SEQ ID NO: 43 - protein, 6 aa, Human papillomavirus
QLNDGP

SEQ ID NO: 44 - protein, 9 aa, Human papillomavirus
FCCKCDSTL

SEQ ID NO: 45 - protein, 9 aa, Human papillomavirus
NIVTFCCKC

SEQ ID NO: 46 - protein, 10 aa, Human papillomavirus
TFCCKCDSTL

SEQ ID NO: 47 - protein, 10 aa, Human papillomavirus
VTFCCKCDST

SEQ ID NO: 48 - protein, 9 aa, Human papillomavirus
DRAHYNIVT

SEQ ID NO: 49 - protein, 9 aa, Human papillomavirus
RAHYNIVTF

SEQ ID NO: 50 - protein, 9 aa, Human papillomavirus
EPDRAHYNI

SEQ ID NO: 51 - protein, 9 aa, Human papillomavirus
VTFCCKCDS

SEQ ID NO: 52 - protein, 10 aa, Human papillomavirus
EPDRAHYNIV

SEQ ID NO: 53 - protein, 10 aa, Human papillomavirus
DRAHYNIVTF

SEQ ID NO: 54 - protein, 9 aa, Human papillomavirus
YNIVTFCCK

SEQ ID NO: 55 - protein, 10 aa, Human papillomavirus
IVTFCCKCDS

SEQ ID NO: 56 - protein, 9 aa, Human papillomavirus
AHYNIVTFC

SEQ ID NO: 57 - protein, 8 aa, Human papillomavirus
DRAHYNIV

SEQ ID NO: 58 - protein, 8 aa, Human papillomavirus
AHYNIVTF

SEQ ID NO: 59 - protein, 8 aa, Human papillomavirus
CCKCDSTL

SEQ ID NO: 60 - protein, 8 aa, Human papillomavirus
PDRAHYNI

SEQ ID NO: 61 - protein, 15 aa, Human papillomavirus
RAHYNIVTFCCKCDS

SEQ ID NO: 62 - protein, 15 aa, Human papillomavirus
DRAHYNIVTFCCKCD

SEQ ID NO: 63 - protein, 15 aa, Human papillomavirus
HYNIVTFCCKCDSTL

SEQ ID NO: 64 - protein, 15 aa, Human papillomavirus
EPDRAHYNIVTFCCK

SEQ ID NO: 65 - protein, 15 aa, Human papillomavirus
ETTDLYCYEQLNDSS

SEQ ID NO: 66 - protein, 14 aa, Human papillomavirus
DEIDGPGQAEPDRA

SEQ ID NO: 67 - protein, 15 aa, Human papillomavirus
CKCDSTLRLCVQSTH

SEQ ID NO: 68 - protein, 15 aa, Human papillomavirus
THVDIRTLEDLLMGT

SEQ ID NO: 69 - protein, 15 aa, Human papillomavirus
HGDTPTLHEYMLDLQ

SEQ ID NO: 70 - protein, 15 aa, Human papillomavirus
EQLNDSSEEEDEIDG

SEQ ID NO: 71 - protein, 15 aa, Human papillomavirus
MGTLGIVCPICSQKP

SEQ ID NO: 72 - protein, 15 aa, Human papillomavirus
YMLDLQPETTDLYCY

SEQ ID NO: 73 - protein, 15 aa, Human papillomavirus
EEEDEIDGPAGQAEP

SEQ ID NO: 74 - protein, 15 aa, Human papillomavirus
AEPDRAHYNIVTFCC

SEQ ID NO: 75 - protein, 15 aa, Human papillomavirus
TLEDLLMGTLGIVCP

SEQ ID NO: 76 - protein, 15 aa, Human papillomavirus
YEQLNDSSEEEDEID

SEQ ID NO: 77 - protein, 15 aa, Human papillomavirus
RLCVQSTHVDIRTLE

SEQ ID NO: 78 - protein, 15 aa, Human papillomavirus
HVDIRTLEDLLMGTL

SEQ ID NO: 79 - protein, 15 aa, Human papillomavirus
IRTLEDLLMGTLGIV

SEQ ID NO: 80 - protein, 15 aa, Human papillomavirus
EYMLDLQPETTDLYC

SEQ ID NO: 81 - protein, 15 aa, Human papillomavirus
TTDLYCYEQLNDSSE

SEQ ID NO: 82 - protein, 15 aa, Human papillomavirus
EDEIDGPAGQAEPDR

SEQ ID NO: 83 - protein, 15 aa, Human papillomavirus
DSTLRLCVQSTHVDI

SEQ ID NO: 84 - protein, 15 aa, Human papillomavirus
EDLLMGTLGIVCPIC

SEQ ID NO: 85 - protein, 15 aa, Human papillomavirus
LYCYEQLNDSSEEED

SEQ ID NO: 86 - protein, 15 aa, Human papillomavirus
IDGPAGQAEPDRAHY

SEQ ID NO: 87 - protein, 15 aa, Human papillomavirus
TDLYCYEQLNDSSEE

SEQ ID NO: 88 - protein, 14 aa, Human papillomavirus
EEDEIDGPGQAEPD

SEQ ID NO: 89 - protein, 15 aa, Human papillomavirus
CDSTLRLCVQSTHVD

SEQ ID NO: 90 - protein, 15 aa, Human papillomavirus
CVQSTHVDIRTLEDL

SEQ ID NO: 91 - protein, 15 aa, Human papillomavirus
DGPAGQAEPDRAHYN

SEQ ID NO: 92 - protein, 15 aa, Human papillomavirus
TLHEYMLDLQPETTD

SEQ ID NO: 93 - protein, 15 aa, Human papillomavirus
KCDSTLRLCVQSTHV

SEQ ID NO: 94 - protein, 15 aa, Human papillomavirus
STLRLCVQSTHVDIR

SEQ ID NO: 95 - protein, 15 aa, Human papillomavirus
RTLEDLLMGTLGIVC

SEQ ID NO: 96 - protein, 15 aa, Human papillomavirus
IVTFCCKCDSTLRLC

SEQ ID NO: 97 - protein, 15 aa, Human papillomavirus
STHVDIRTLEDLLMG

SEQ ID NO: 98 - protein, 6 aa, Human papillomavirus
AHYNIV

SEQ ID NO: 99 - protein, 6 aa, Human papillomavirus
HYNIVT

SEQ ID NO: 100 - protein, 6 aa, Human papillomavirus
YNIVTF

SEQ ID NO: 101 - protein, 6 aa, Human papillomavirus
NIVTFC

SEQ ID NO: 102 - protein, 6 aa, Human papillomavirus
IVTFCC

SEQ ID NO: 103 - protein, 6 aa, Human papillomavirus
VTFCCK

SEQ ID NO: 104 - protein, 6 aa, Human papillomavirus
TFCCKC

SEQ ID NO: 105 - protein, 6 aa, Human papillomavirus
FCCKCD

SEQ ID NO: 106 - protein, 21 aa, Human papillomavirus
EQLNDSSEEEDEIDGPAGQAE

SEQ ID NO: 107 - protein, 21 aa, Human papillomavirus
AEPDRAHYNIVTFCCKCDSTL

SEQ ID NO: 108 - protein, 10 aa, Human papillomavirus
YNIVTFCCKC

SEQ ID NO: 109 - protein, 4 aa, Homo sapiens
RGDS

SEQ ID NO: 110 - protein, 5 aa, Homo sapiens
KFERD

SEQ ID NO: 111 - protein, 4 aa, Homo sapiens
KDEL

SEQ ID NO: 112 - protein, 7 aa, Human papillomavirus
QLNDSSE

SEQ ID NO: 113 - protein, 7 aa, Human papillomavirus
SSEEEDE

(19) United States
(12) Patent Application Publication
(10) Pub. No.: US 2007/0111261 A1
(43) Pub. Date: May 17, 2007

(54) Antigens

(75) Inventors: Abraham Mittelman, Scarsdale, NY (US); Darja Kanduc, Bari (IT)

Correspondence Address: KENYON & KENYON LLP, ONE BROADWAY, NEW YORK, NY, 10004, US

(73) Assignee: Applied Immune Technologies, Inc., Scarsdale, NY (US)

(21) Appl. No.: 11/635,458

(22) Filed: Dec. 6, 2006

Related U.S. Application Data
(63) Continuation of application No. 10/648,547, filed on Aug. 25, 2003, now abandoned.
Application No. 10/648,547 is a continuation of application No. 10/306,541, filed on Nov. 25, 2002, now abandoned.
(60) Provisional application No. 60/333,249, filed on Nov. 23, 2001.

(51) Int. Cl. G01N 33/53; C07H 21/04; C12P 21/06; C07K 14/74

(52) U.S. Cl. 435/7.1; 435/69.1; 435/320.1; 435/325; 530/350; 536/23.5

(57) ABSTRACT

The invention provides a method for identifying amino acid sequences in antigens of interest that are useful for evoking immune responses. The amino acid sequences have low sequence similarity to the host proteome and are predicted to bind to MHC. Also disclosed are HPV epitopes that evoke Class I or Class II mediated immune responses.

### RELATED APPLICATIONS

This application is a continuation of U.S. application Ser. No. 10/648,547, filed Aug. 25, 2003, which is a continuation of U.S. application Ser. No. 10/306,541, filed Nov. 25, 2002, now abandoned, and claims priority from provisional U.S. Application Ser. No. 60/333,249, filed Nov. 23, 2001, incorporated herein by reference in its entirety.

### BACKGROUND OF THE INVENTION

Molecular mimicry has been studied as a phenomenon underlying autoimmune responses and diseases. When linear and/or conformational amino acid sequences are shared by microbial/viral agents and ‘self’ molecules, autoimmunity may occur if the host immune response against the infectious agents cross-reacts with host ‘self’ sequences. The ability of the immune system to distinguish between self and non-self molecules is an important property in maintaining tissue/organism integrity. Breakage of this self-tolerance is one of the main bases for autoimmune diseases. Molecular mimicry induced autoimmunity often occurs when the non-self and host determinants are similar enough to cross-react, yet different enough to break immunological tolerance.

When high degrees of similarity are present between non-self and self molecules, the breaking of the powerful self-tolerance mechanisms that avoid harmful self-reactivity seems less likely. Therefore, sharing of epitopes of high similarity with the host's molecules may represent a viral characteristic evolved to escape immune surveillance. The tolerance mechanisms used to prevent autoimmune destruction could be the main basis through which tumor-associated antigens and antigens associated with infectious agents escape from functional antigen-specific immune recognition.

For example, human papilloma viruses (HPV) are viruses of low immunogenicity. Epidemiological data indicate that sexually transmitted HPV is an important aetiological agent in the development of cervical cancer, which causes 15% of deaths from cancer in women worldwide. Studies have demonstrated that the proliferation and malignant phenotype of human cervical carcinoma cell culture depends on continuous expression of HPV oncogenes E6 and E7. Consequently, great efforts have been directed towards designing therapeutic vaccines against HPV-induced cervical carcinoma using the HPV16/18 E6 and E7 tumor-associated antigens as targets.

The success of HPV infection is due in part to avoidance of the host's immune surveillance system that would otherwise respond to the foreign viral oncoproteins and stem the spread of HPV infection. One reason for the failure of the immune system to control HPV infection and for the failure of E6 and/or E7 based vaccines may reside in the poor antigenicity, that is, poor non-self character, of the viral peptides presented by the MHC.

Likewise, the similarity of tumor associated antigens, i.e., self antigens, to the human proteome presents a significant hurdle in the development of cancer vaccines. Theoretically, an effective anti-cancer vaccine should contain antigenic sequences effective to stimulate an immune response, but methods for identification of such effective sequences have not been forthcoming.

Active fields of study in vaccine development include antigen processing, peptide availability, analysis of structural features of peptides, binding to histocompatability molecules, and polymorphism of histocompatibility molecules. On the basis of increasing knowledge of the nature of MHC-peptide interaction and T cell receptor recognition, algorithms have been developed to predict epitopic peptides. However, it is difficult to find relevance in the epitopic sequences that have been reported to date.

### SUMMARY OF THE INVENTION

The present invention provides a method of identifying epitopes which are useful for evoking immune responses against an antigen of interest. Significantly, the method is particularly advantageous for identifying useful immunogenic epitopes in antigens of interest that otherwise have poor immunogenicity. The antigen of interest can be, for example, a tumor antigen, or an antigen from an infectious agent. According to the invention, useful epitopes can be identified which bind effectively to class I and/or class II major histocompatibility complex (MHC) and which have amino acid sequences that are under-represented in host proteins.

The basis of the invention is the discovery that antigens which have low immunogenicity display the greatest sequence similarity to the host proteome. The sequence similarity is evident when short segments of the antigen are compared to host proteome sequences. Further, it is demonstrated that a mouse antibody raised against a full length viral oncoprotein of poor immunogenicity binds to a determinant having both high MHC II binding potential and a low level of similarity to the mouse proteome. That is, effective immunogenic peptides tend to be under-represented in the host's proteome.

Accordingly, an aspect of the invention is a method for identifying an immunodominant epitope of an antigen by examining amino acid sequences within the antigen for binding affinity to an MHC molecule, examining amino acid sequences within the antigen to determine sequence similarity to the host proteome, and selecting an amino acid sequence within the antigen predicted to have high MHC binding affinity and low sequence similarity to the host proteome.

In one embodiment, the MHC molecule is selected to be a class I MHC molecule. In another embodiment, the MHC molecule is selected to be a class II MHC molecule. In certain embodiments, it may be preferred to identify an amino acid sequence that binds to more than one MHC molecule. The MHC binding sequences may be adjacent of overlapping. In an embodiment of the invention, MHC binding is predicted by comparing amino acid sequences within the antigen to a consensus MHC binding sequence. Such a comparison may be performed manually of with the aid of a computer-driven algorithm.

According to the invention, amino acid sequence similarity between the antigen and the host proteome is examined by examining short amino acid sequences within the antigen and comparing them to the host proteome. The amino acid sequences are preferably overlapping, and generally 20 amino acids or less. In a preferred embodiment, the overlapping sequences are 4 to 10 amino acids in length, and more preferably 5, 6, or 7 amino acids in length. To insure that the sequence comparison has sufficient resolution, the overlapping amino acid sequences are preferentially offset by a small number of amino acids. In an preferred embodiment of the invention, sequential overlapping sequences are evaluated that are offset by 5 amino acids. In a more preferred embodiment, the offset is one of two amino acids.

The invention is further directed to a method of producing a polypeptide useful for eliciting an immune response against an antigen in a host comprising analyzing amino acid sequences within the antigen for binding affinity to an MHC molecule, examining amino acid sequences within the antigen to determine sequence similarity to the host proteome, selecting an amino acid sequence having high MHC binding affinity and low sequence similarity, and producing a polypeptide comprising the selected amino acid sequence.

The invention provides a method of eliciting a therapeutic immune response to an antigen comprising administering to a host an immunologically effective amount a polypeptide comprising an amino acid sequence identified by analyzing amino acid sequences within the antigen for binding affinity to an MHC molecule, examining amino acid sequences within the antigen to determine sequence similarity to the host proteome, and selecting an amino acid sequence having high MHC binding affinity and low sequence similarity. In one embodiment, the antigen is a tumor antigen. In another embodiment, the antigen is from an infectious agent. In a further embodiment, the administered polypeptide comprises a B cell epitope as well as an epitope selected to have affinity for MHC.

The present invention provides a rapid and powerful method for identifying peptides for use in immunogenic compositions. Peptides identified by the method comprise antigenic determinants which can induce immune responses against antigens, for example, cancer antigens and infectious agents, and are particularly useful for inducing immune responses against antigens which are otherwise known or found to be poorly immunogenic.

### BRIEF DESCRIPTION OF THE DRAWINGS

FIG. 1 shows plots of sequence matches between human proteins and 5-mer amino acid sequences derived from (a) E7 oncoprotein, (b) SV40 small t antigen, (c) Newcastle disease virus haemagglutinin-neuramidase polypeptide fragments and (d) yellow fever virus NS2A protein sequence.

FIG. 2 shows the identification of 15-mer polypeptides recognized by mouse anti-HPV16 E7 mAb ED17 by dot immunoassay. Peptide: 1) control: E7₂₅₋₃₉ YEQLNDSSEEEDEID (SEQ ID NO:76); 2) E7₈₄₋₉₈ MGTLGIVCPICSQKP (SEQ ID NO:71); 3) E7₂₋₁₆ HGDTPTLHEYMLDLQ (SEQ ID NO:69); 4) E7₄₉₋₆₃ RAHYNIVTFCCKCDS (SEQ ID NO:61); 5) E7₃₂₋₄₆ SEEEDEIDGPAGQAE (SEQ ID NO:39).

FIG. 3 shows epitope scanning by dot immunoassay for identification of the epitope from E7₄₉₋₆₃ RAHYNIVTFCCKCDS (SEQ ID NO:61) recognized by mouse anti-HPV16 E7 mAb ED17. Peptide: 1)AHYNIV (SEQ ID NO:98); 2) HYNIVT (SEQ ID NO:99); 3) YNIVTF (SEQ ID NO:100); 4) NIVTFC (SEQ ID NO:101); 5) IVTFCC (SEQ ID NO:102); 6) VTFCCK (SEQ ID NO:103); 7) TFCCKC (SEQ ID NO:104); 8) FCCKCD (SEQ ID NO: 105).

### DETAILED DESCRIPTION OF THE INVENTION

The present invention is directed to rapid evaluation of antigens to identify regions that are of immunological interest. According to the present invention, antigens are examined to identify sequences having improved immunogenicity, not just on the basis of MHC or antibody binding, but also on the basis that they will be recognized as foreign, rather than self antigens. Accordingly, the invention is directed to identification of immunodominant epitopes, and the use of polypeptides displaying immunodominant epitopes for eliciting desired immune responses. In certain embodiments, immunodominant epitopes may be selected in view of a subject's MHC makeup. In other embodiments, immunogenic portions of antigens that are otherwise poorly immunogenic can be identified and used as therapeutic candidates. Thus, the present invention can be used to identify sequences of amino acids which are useful for inducing host immune responses against antigens of interest, particularly cancer antigens and antigens from infectious agents, including antigens which may be seen as self and be poorly immunogenic. According to the method, an antigen is analyzed to identify regions of interest that are both capable of binding to class I or class II MHC, and under-represented in the host proteome.

In general, specific binding of antigenic peptides to MHC is a prerequisite for immunologic reactivity/anergy. Peptide sequences that trigger immune cell activation are classified as immunodominant epitopes, whereas determinants that fail to elicit any response are called cryptic. The invention is based on the discovery that, in order to identify immunologically important epitopes, and thus immunologically useful peptides, it is necessary to consider not only strength of MHC binding, but also molecular mimicry phenomena. Immunogenicity and lack thereof is also controlled by the similarity between an antigen and the self proteome. For example, the non-immunogenicity of tumor associated antigens and viral oncoproteins can be explained by high levels of similarity of the antigens and oncoproteins to self sequences.

Accordingly, the invention enables the identification of polypeptides having motifs that are absent or scarcely represented in endogenous self-proteins. Such polypeptides are especially useful for inducing immune responses against antigens that otherwise have a high similarity to self proteins. Accordingly the polypeptides may be used to elicit an immune response to tumor and infectious disease antigens that are themselves poorly immunogenic.

MHC binding is evaluated, for example, by predictions based on MHC-peptide binding scoring methods or MHC-binding sequence motifs to identify peptide sequences that are likely to be ercognized by the immune system. For example, the SYFPEITHI database (Ramensee et al., 1999, Immunogenetics 50:213-19) contains information on peptide sequences, anchor positions and MHC specificity for peptides that bind to class I and class II MHC and provides an epitope prediction algorithm (Rammensee et al., Immunogenetics 1999, 50:213-219). An alternative approach is the weight matrix approach in which weights for each of the amino acid residues in every position along a peptide can be generated for a given MHC allele, based on experimental binding data for large ensembles of sequence variants. Peptide sequences from the antigen of interest are assigned scores based on their sequence and the matrix for the appropriate MHC allele. In other cases, such as where an MHC structure is available, peptides can be “threaded” through the structural model to obtain an estimate of the binding energy of a peptide in the MHC groove. It will be apparent that, in many cases where B cell epitopes are sought, they will overlap or fall within MHC binding sequences, since the method generally identifies polypeptides with MHC binding ability.

MHC binding can also be confirmed by biochemical and physical measurements, such as by measurement of affinity by direct binding or competitive assays, nuclear magnetic resonance (NMR) and the like.

Sequence similarity between the antigen of interest and the host proteome can be evaluated by any convenient method designed to analyze portions of the amino acid sequence of the antigen. That is, sequence comparisons are not made using the entire amino acid sequence of the antigen of interest at once, but by using smaller portions, the size of which my be chosen to be on the order of a T cell or a B cell epitope. The entire amino acid sequence can of course be analyzed, but taking smaller portions at a time. The goal is to identify portions of the antigen corresponding to a T cell or B cell epitope that have affinity for class I or class II MHC, and have amino acid sequences that are dissimilar from the host proteome. Dissimilarity is determined based on a sliding window of a few amino acids, rather than over the antigen as a whole. For example, in an embodiment where it is desired to identify an immunogenic MHC binding epitope of, for example, 12 amino acids, 12 amino acid sequences identified as having MHC specific motifs would then be compared to the host proteome not as 12 amino acid sequences, but as individual overlapping sequences of, for example, 5, 6 or 7 amino acids. Ideally, the overlapping sequences are offset by just a few amino acids at most.

Thus, sequence similarity of an antigen to a host proteome is evaluated by dissecting the antigen of interest into short overlapping peptide sequences, each of which is evaluated for similarity to host proteins. In an embodiment of the invention, the overlapping peptide “probe” sequences are 4 to 10 amino acids in length. In a preferred embodiment, the sequence probes are 7, 6 or 5 amino acids and overlap by 1 or 2 amino acids.

In general, comparisons of an antigen and a host proteome are made using computer based methods. Sequence sources and sequence similarity analysis methods that can be used for such comparisons include for example, the NCBI, SWISS-PROT, and PIR protein and nucleotide sequence databases (including human, microbial and other eukaryotic genomes), and PRINTS, FASTA, BLAST, and other computer algorithms known in the art. See, for example, Junker et al., 2000, J. Biotechnol. 78:221-34; McGarvey et al., 2000, Bioinformatics, 16:290-1; Pearson, 2000, Methods Mol. Biol. 132:185-219; Scordis et al., 1999, Bioinformatics 15:799-806; Wheeler et al., 2000, Nucleic Acids Res. 28:10-4.

Similarity is evaluated with respect to several host proteins. The number of proteins need not be large. For example, in one experiment, data was obtained by comparison to the SWISS-PROT database that showed high similarity between HPV16 E7 and human proteins involved in a number of critical regulatory processes. Some human proteins were found to contain multiple identical or different E7 peptide motifs. The antigen-proteome similarity became evident on the basis of only a subset of the entire (accessible) human proteome.

The method is compatible with the avoidance of sequence motifs that have important biological functions. This is because such motifs are well represented in the proteome of the host. Examples include RGDS (SEQ ID NO:109), KFERD (SEQ ID NO:110), and KDEL (SEQ ID NO:111) motifs which are signals for integrin binding, lysosomal targeting, and endoplasmic reticulum retention, respectively.

The method is applicable to any antigen of interest. Antigens of particular interest are associated with a cancer or neoplastic disease, such as, for example, sarcoma, lymphoma, leukemia, carcinoma and melanoma. In other embodiments, the antigen can be from an infectious agent, such as, for example, a bacterium, a virus, a mycoplasma, a fungus and the like. A self antigen, such as might be expressed or overexpressed by a neoplastic cell, is analyzed in the same manner as a poorly immunogenic foreign antigen, to identify portions that are poorly represented in the host proteome. With respect to cancer antigens, certain self antigens can be particularly attractive targets is they are express in a developmental or cell type specific manner.

Immunodominant epitopes identified according to the invention can be used for therapeutic purposes. The invention provides vaccine strategies based on peptides having amino acid sequences that are under-represented in a host. For example, as disclosed in Example 3, there is often a correspondence between peptides that have affinity for class II MHC molecules and B cell epitopes. That is, the class II binding sequence often comprises the B cell epitope. As provided below, a polypeptide comprising amino acids 44-62 of HPV E7 protein has very low similarity to human proteins and comprises the binding site for an E7-binding MAb. Alternatively, a B cell epitope can be joined to a class II binding sequence identified by the invention. For example, an unshared epitope from the same HPV E7₄₄₋₆₂ peptide has been shown to promote strong antibody responses when linked to other B cell epitopes of E7. Moreover, the HPV E7₄₄₋₆₂ peptide is effective for preventing outgrowth of HPV-transformed tumor cells in mice. Accordingly, the invention provides MHC binding polypeptides that are particularly useful for eliciting immune responses, either by themselves, or when conjugated to other antigens.

The invention can also be used to redirect immune responses against particular portions of an antigen of interest. For example, several systemic rheumatic diseases have been demonstrated to be associated with infection. The associations include that of hepatitis B infection with systemic necrotizing vasculitis (polyarteritis nodosa), hepatitis C infection with IgG-IgM cryoglobulinemia, and the documentation that an epidemic form of arthritis, primarily in children, is caused by infection with a previously unidentified spirochete Borrelia burgdorferi. Mycoplasma has on occasion been suspected to be a trigger. Autoantibodies frequently found in patients with rheumatic illness parallel antibodies that occur in a variety of infectious illnesses. The identification of potential microbial triggering agents for the reactive arthritis and for the spondyloarthropathies and a demonstration of the potential molecular relationships between the HLA B27 histocompatibility antigen and certain enteric pathogens gives further support to the hypothesis that infection triggers rheumatic and other autoimmune diseases.

According to the present invention, it is now possible to identify new and useful antigenic determinants in such infectious organisms. Such determinants, which might not be immunodominant in their usual context, can be used to elicit immune responses directed at the organism, and not at immunogenic determinants common to the organism and a self-antigen. A composition comprising a new antigenic determinant identified according to the invention is used to treat the rheumatoid or autoimmune disease. Alternatively, the composition is used to immunize a subject against the infectious agents associated with the disease. Immunization can be especially useful where a relationship has been identified between the disease and an HLA type.

Immunogenic peptides identified by the method can be relatively short. As is well known in the art, short linear peptides can be used to induce useful immune responses, and a peptide used for immunization may be limited to a single T cell or B cell epitope. Alternatively, the antigenic peptides can be incorporated into longer sequences of amino acids. The additional sequences can, for example, be native to the protein from which the peptide antigen is selected, or sequences that confer some other function, such as the ability to bind to a heat shock protein. In certain embodiments, tandem arrays will be produced which comprise multiple copies of the antigenic peptide, or mixtures of two or more antigenic peptides selected from the same antigen of interest.

Immunogenic compositions comprising antigenic peptides identified according to the invention may be administered to a subject using either a protein or nucleic acid vaccine so as to produce in the subject, an amount of the selected peptide which is effective in inducing a therapeutic immune response in the subject. The subject may be a human or nonhuman subject. The term “therapeutic immune response”, as used herein, refers to an increase in humoral and/or cellular immunity, as measured by standard techniques, which is directed toward the antigen of interest. Preferably, the induced level of immunity directed toward the antigen is at least four times, and preferably at least 16-fold greater than the levels of the immunity directed toward antigen prior to the administration of the compositions of this invention. The immune response may also be measured qualitatively, wherein by means of a suitable in vitro assay or in vivo an arrest in progression or a remission of neoplastic or infectious disease in the subject is considered to indicate the induction of a therapeutic immune response.

Compositions comprising antigenic peptides of the invention may be administered cutaneously, subcutaneously, intravenously, intramuscularly, parenterally, intrapulmonarily, intravaginally, intrarectally, nasally or topically. The composition may be delivered by injection, particle bombardment, orally or by aerosol.

Compositions for administration may further include various additional materials, such as a pharmaceutically acceptable carrier. Suitable carriers include any of the standard pharmaceutically accepted carriers, such as phosphate buffered saline solution, water, emulsions such as an oil/water emulsion or a triglyceride emulsion, various types of wetting agents, tablets, coated tablets and capsules. Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients. The composition of the invention may also include suitable diluents, preservatives, solubilizers, emulsifiers, adjuvants and/or carriers. Such compositions may be in the form of liquid or lyophilized or otherwise dried formulations and may include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g. glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexing with metal ions, or incorporation of the material into or onto particulate preparations of polymeric compounds such as polylactic acid, polyglycolic acid, hydrogels, etc. or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

As an alternative to direct administration of the heat shock protein and target antigen, one or more poly-nucleotide constructs may be administered which encode heat shock protein and target antigen in expressible form. The expressible polynucleotide constructs are introduced into cells in the subject using ex vivo or in vivo methods. Suitable methods include injection directly into tissue and tumors, transfecting using liposomes, receptor-mediated endocytosis, particle bombardment-mediated gene transfer, and other methods of gene transfer. The polynucleotide vaccine may also be introduced into suitable cells in vitro which are then introduced into the subject. To construct an expressible polynucleotide, a region encoding the peptide antigen is prepared and inserted into a mammalian expression vector operatively linked to a suitable promoter such as the SV40 promoter, the cytomegalovirus (CMV) promoter, or the Rous sarcoma virus (RSV) promoter. The resulting construct may then be used as a vaccine for genetic immunization. The nucleic acid polymer(s) could also be cloned into a viral vector. Suitable vectors include but are not limited to retroviral vectors, adenovirus vectors, vaccinia virus vectors, pox virus vectors and adenovirus-associated vectors. Specific vectors which are suitable for use in the present invention are pCDNA3 (In-Vitrogen), plasmid AH5 (which contains the SV40 origin and the adenovirus major late promoter), pRC/CMV (In Vitrogen), pCMU II (Paabo et al., EMBO J. 5:1921-1927 (1986)), pZip-Neo SV (Cepko et al., Cell 37:1053-1062 (1984)) and pSRα (DNAX, Palo Alto, Calif.).

It is to be understood and expected that variations in the principles of invention herein disclosed may be made by one skilled in the art and it is intended that such modifications are to be included within the scope of the present invention.

The examples which follow further illustrate the invention, but should not be construed to limit the scope in any way.

Natale et al. (2000) Immunol. Cell Biol. 78:580-585 and all other references mentioned herein are incorporated by reference in their entirety.

### EXAMPLES

### Example 1

To investigate the molecular mimicry between the HPV16 E7 oncoprotein sequence and human proteome, a systematic study of sequence similarity was done by dissecting the E7 oncoprotein sequence into 7, 6, and 5 aa motifs that were used as sequence probes. The analyzed HPV16 E7 oncoprotein sequence was as reported by Seedorf et al. (Medline accession no. K02718). Sequence similarity analyses were conducted by using the MEDLINE, FASTA, BLAST, PIR, SWISS-PROT and PRINTS sequence analysis programs. The SYFPEITHI program (Rammensee, H.-G., et al., 1999) was used as database of HLA ligands and peptide motifs.

As controls, the sequences of the following proteins were analyzed: (i) small t antigen (SWISS-PROT accession no. P03081) from simian virus 40 (SV40); (ii) the non-structural protein NS2A (Medline U89339) from yellow fever virus (YFV); and (iii) three fragments from the haemagglutinin-neuramidase (HN) protein (EMBL accession no. X79092) from Newcastle disease virus (NDV).

Sequences from the NDV HN protein were examined because of the high immunogenic potential shown by the ssRNA NDV. In fact, it has repeatedly reported that treatment with lysates of NDV-infected allogeneic human tumor is able to elicit humoral immune responses against tumour cell-associated antigens, thus breaking the tumor immune tolerance. Three polypeptide fragments from the haemagglutinin-neuramidase protein were approximately 33aa long each, for a total of 100 aa, and were spaced at almost regular intervals along the entire protein sequence. The fragments were: aa 176-208 (fragment 1); aa 337-369 (fragment 2); and aa 467-499 (fragment 3).

The NS2A sequence from the YFV was examined, as seroepidemiological surveys in African populations have shown some seropositivity for YFV antibodies, thus indicating the ability of this ssRNA virus to elicit an antibody response.

A low degree of similarity to human protein sequences was expected for YFV NS2A and NDV HN protein sequences compared with HPV16 E7. The cell growth regulatory small t antigen from the dsDNA virus SV40 was also analyzed in order to have a genome/function-based control, as HPV16 is a dsDNA and E7 a growth regulatory protein.

By using 7-mer sequence probes, it was found that the E7 protein 7 aa motif QLNDSSE (SEQ ID NO: 112) gives one human match corresponding to Na+/Pi transport protein 4 (SwissProt O00476). The E7 SSEEEDE (SEQ ID NO:113) motif is present in xeroderma pigmentosum group G (XP-G) complementing protein (SwissProt P28715). The same motif is also present in retinoblastoma binding protein 1 (RBBP-1; SwissProt P29374), a critical cell-cycle regulatory protein. In contrast, no human polypeptide has 7-mer motifs in common with the control SV40 small t antigen, NDV HN or YFV NS2A proteins.

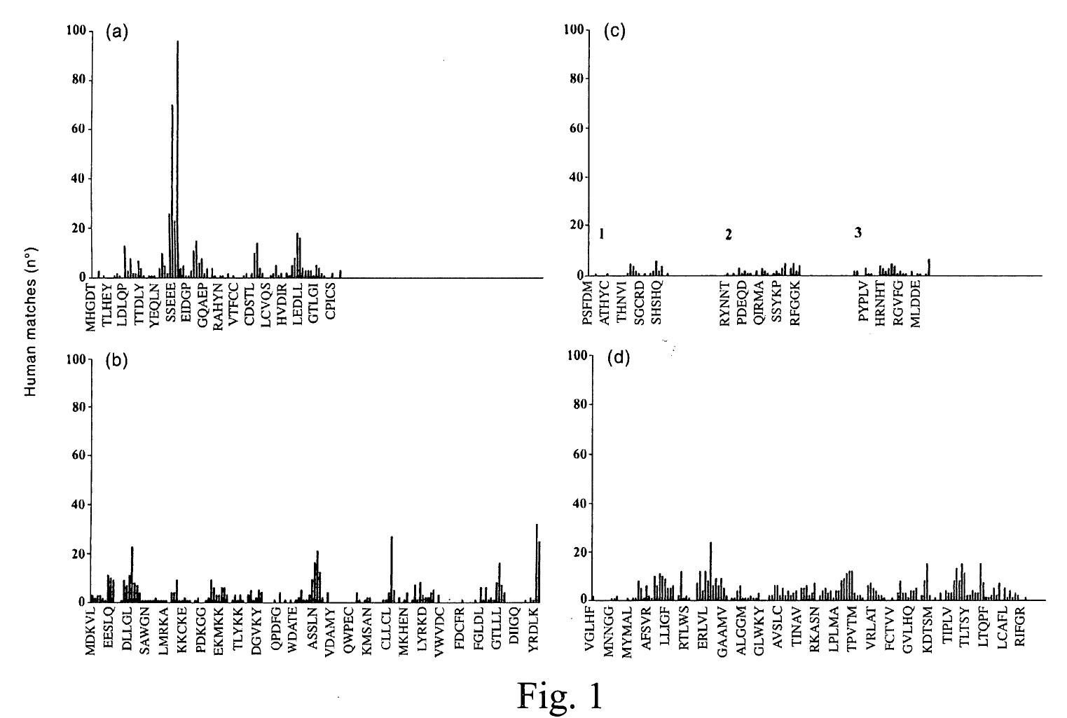

These data provided the incentive for a thorough analysis of E7 motifs present in the human proteome. Because 5-6 aa are the minimum requisite to induce an antibody response, the oncoprotein sequence and the control sequences were dissected into 5-mer motifs that were used as sequence probes. FIG. 1 illustrates the similarity sequence data obtained. It can be seen that all four proteins examined here present motifs in common with the human proteome. However, the highest number of matches was found in the E7 oncoprotein sequence (FIG. 1a). The SV40 small t antigen sequence showed similarity to 5-mer portions of a number of human proteins (FIG. 1b), suggesting the tendency of dsDNA viruses to ‘borrow’ genetic information and, consequently, sequence similarity from their hosts. At the same time, it is evident that long viral sequences in SV40 small t antigen have no matches at all to human proteome, thus offering possible epitopic determinants unknown to the host. The three HN control fragments from the immunogenic NDV had the lowest number of human matches (FIG. 1c). YFV NS2A also showed fewer human matches than E7 oncoprotein (FIG. 1d).

Further computer-assisted analysis showed that a number of human proteins harbored multiple HPV16 E7 4-mer motifs of both identical and different peptide sequences. Three examples are reported in Table 1.

TABLE 1
Identical and different multiple E7 peptide motifs
in human proteins

| | | |
|---|---|---|
| Amino acid position | Motif | SEQ ID NO: |

Collagen alpha-1 (V) chain precursor*

| | | |
|---|---|---|
| 475 | GPAG | 1 |
| 559 | GPAG | 1 |
| 601 | GPAG | 1 |
| 940 | GPAG | 1 |
| 1042 | GPAG | 1 |
| 1084 | GPAG | 1 |
| 1093 | GPAG | 1 |
| 1114 | GPAG | 1 |
| 1129 | GPAG | 1 |
| 1144 | GPAG | 1 |
| 1354 | GPAG | 1 |
| 1396 | GPAG | 1 |

Cell proliferation-associated antigen of
antibody Ki-67 †

| | | |
|---|---|---|
| 1010 | LQPE | 2 |
| 1099 | LEDL | 3 |
| 1138 | DTPT | 4 |
| 1221 | LEDL | 3 |
| 1260 | DTPT | 4 |
| 1343 | LEDL | 3 |
| 1382 | DTPT | 4 |
| 1464 | LEDL | 3 |
| 1502 | DTPT | 4 |
| 1585 | LEDL | 3 |
| 1746 | DTPT | 4 |
| 1868 | DTPT | 4 |
| 1951 | LEDL | 3 |
| 2073 | LEDL | 3 |
| 2112 | DTPT | 4 |
| 2191 | LEDL | 3 |
| 2313 | LEDL | 3 |
| 2434 | LEDL | 3 |
| 2556 | LEDL | 3 |
| 2628 | QSTH | 5 |
| 2676 | LEDL | 3 |
| 2748 | ETTD | 6 |
| 2915 | LEDL | 3 |

Titin, cardiac muscle ‡

| | | |
|---|---|---|
| 748 | TTDL | 7 |
| 4317 | LNDS | 8 |
| 6233 | EEED | 9 |
| 8358 | STLR | 10 |
| 10,321 | PTLH | 11 |
| 10,738 | TLRL | 12 |
| 15,301 | EEDE | 13 |
| 15,380 | TLRL | 12 |
| 18,203 | DEID | 14 |
| 18,627 | TLRL | 12 |
| 20,427 | TTDL | 7 |
| 23,345 | DEID | 14 |
| 24,147 | STLR | 10 |
| 24,148 | TLRL | 12 |
| 25,020 | IRTL | 15 |
| 25,293 | DSTL | 16 |
| 25,294 | STLR | 10 |

To determine the immunological potencies of shared and unshared peptide sequences, the ability of E7 sequences determined to be similar or dissimilar to human proteins to bind HLA molecules was examined. Two E7 fragments: EQLNDSSEEEDEIDGPAGQAE (aa 26-46; SEQ ID NO:106), which has a high level of similarity to the human proteome (total number of 5-mer human matches, 290), and AEPDRAHYNIVTFCCKCDSTL (aa 45-65; SEQ ID NO:107), which has a low level of similarity to the human proteome (total number of 5-mer human matches, 14; see FIG. 1, were analyzed. The two fragments were analyzed for potential T-cell epitopes taking into consideration the amino acids in the anchor and auxiliary anchor positions by using SYFPEITHI program. In this program, the HLA-binding potential score is calculated by giving the amino acids of a certain peptide a specific value depending on whether they are anchor, auxiliary anchor or preferred residues. Amino acids that are regarded as having a negative effect on the binding ability are also evaluated by a negative value. Table 2 illustrates the data obtained by submitting the two E7 viral polypeptide sequences to SYFPEITHI program analysis. On the whole, the table shows that peptides derived from the high-similarity E7 sequence EQLNDSSEEEDEIDGPAGQAE (SEQ ID NO:106) show a general tendency to bind to HLA-A type molecules with higher strength than peptides from the low-similarity E7 polypeptide AEPDRAHYNIVTFCCKCDSTL (SEQ ID NO:107). In contrast, unshared sequences have higher binding potential to HLA-B-type molecules than shared motifs.

TABLE 2
Molecular mimicry level and binding potential to HLA molecules of E7 peptides

| | |
|---|---|
| High-similarity E7 sequence | Low-similarity E7 sequence |

| | | SEQ | | | | SEQ | | |
|---|---|---|---|---|---|---|---|---|
| HLA type | Peptide Sequence | ID NO: | Matches | Score | Peptide Sequence | ID NO: | Matches | Score |
| A*0201 | IDGPAGQA | 17 | 35 | 9 | — | | | |
| | EDEIDGPA | 18 | 10 | 9 | — | | | |
| | QLNDSSEEE | 19 | 113 | 14 | FCCKCDSTL | 44 | 5 | 13 |
| | EIDGPAGQA | 20 | 36 | 12 | NIVTFCCKC | 45 | 1 | 11 |
| | QLNDSSEEED | 21 | 136 | 14 | TFCCKCDSTL | 46 | 5 | 12 |
| | NPSSEEEDEI | 22 | 221 | 10 | VTFCCKCDST | 47 | 3 | 12 |
| A*0203 | IDGPAGQA | 17 | 35 | 8 | — | | | |
| | EDEIDGPA | 18 | 10 | 8 | — | | | |
| | EIDGPAGQA | 20 | 36 | 9 | DRAHYNIVT | 48 | 4 | 3 |
| | EEDEIDGPA | 23 | 14 | 9 | RAHYNIVTF | 49 | 4 | 2 |
| | DEIDGPAGQA | 24 | 37 | 10 | — | | | |
| | EEEDEIDGPA | 25 | 110 | 10 | — | | | |
| A1 | SEEEDEIDG | 26 | 129 | 16 | EPDRAHYNI | 50 | 7 | 10 |
| | SSEEEDEID | 27 | 291 | 16 | VTFCCKCDS | 51 | 3 | 7 |
| | SSEEEDEIDG | 28 | 199 | 20 | EPDRAHYNIV | 52 | 7 | 10 |
| | EIDGPAGQAE | 29 | 44 | 12 | VTFCCKCDST | 47 | 3 | 7 |
| A26 | EIDGPAGQA | 20 | 36 | 20 | RAHYNIVTF | 49 | 4 | 15 |
| | EEEDEIDGP | 30 | 107 | 12 | VTFCCKCDS | 51 | 3 | 12 |
| | EIDGPAGQAE | 29 | 4 | 21 | DRAHYNIVTF | 53 | 4 | 22 |
| | EEDEIDGPAG | 31 | 25 | 11 | TFCCKCDSTL | 46 | 5 | 15 |
| A3 | EIDGPAGQA | 20 | 36 | 17 | RAHYNIVTF | 49 | 4 | 16 |
| | QLNDSSEEE | 19 | 113 | 13 | YNIVTFCCK | 54 | 3 | 13 |
| | EIDGPAGQAE | 29 | 44 | 14 | DRAHYNIVTF | 53 | 4 | 13 |
| | QLNDSSEEED | 21 | 136 | 13 | IVTFCCKCDS | 55 | 4 | 12 |
| B*0702 | — | | | | EPDRAHYNI | 50 | 7 | 18 |
| | — | | | | FCCKCDSTL | 44 | 5 | 11 |
| | EEEDEIDGPA | 25 | 110 | 8 | EPDRAHYNIV | 52 | 7 | 18 |
| | NDSSEEEDEI | 22 | 221 | 8 | TFCCKCDSTL | 46 | 5 | 10 |
| B*1510 | IDGPAGQAE | 17 | 39 | 5 | FCCKCDSTL | 44 | 5 | 12 |
| | EDEIDGPAG | 32 | 21 | 4 | AHYNIVTFC | 56 | 4 | 11 |
| B*2705 | DSSEEEDEI | 33 | 219 | 9 | RAHYNLVTF | 49 | 4 | 19 |
| | EIDGPAGQA | 20 | 36 | 5 | FCCKCDSTL | 44 | 5 | 15 |
| B*2709 | DSSEEEDEI | 33 | 219 | 8 | RAHYNIVTF | 49 | 4 | 13 |
| | EQLNDSSEE | 34 | 47 | 3 | FCCKCDSTL | 44 | 5 | 10 |
| B*5101 | DGPAGQAE | 35 | 40 | 14 | DRAHYNIV | 57 | 2 | 16 |
| | SSEEEDEI | 36 | 193 | 11 | AHYNIVTF | 58 | 3 | 13 |
| | DSSEEEDEI | 33 | 219 | 17 | EPDRAHYNI | 50 | 7 | 20 |
| | DEIDGPAGQ | 37 | 31 | 7 | RAHYNIVTF | 49 | 4 | 19 |
| B8 | SSEEEDEI | 36 | 193 | 10 | CCKCDSTL | 59 | 5 | 20 |
| | EIDGPAGQ | 38 | 30 | 6 | PDRAHYNI | 60 | 3 | 12 |
| | DSSEEEDEI | 33 | 219 | 9 | EPDRAHYNI | 50 | 7 | 14 |
| | QLNDSSEEE | 19 | 113 | 7 | RAHYNIVTF | 49 | 4 | 13 |
| DRB1*0101 | SEEEDEIDGPAGQAE | 39 | 173 | 14 | RAHYNIVTFCCKCDS | 61 | 8 | 21 |
| | QLNDSSEEEDEIDGP | 40 | 243 | 9 | DRAHYNIVTFCCKCD | 62 | 6 | 15 |
| DRB1*0301 | DSSEEEDEIDGPAGQ | 41 | 255 | 12 | HYNIVTFCCKCDSTL | 63 | 8 | 11 |
| (DR17) | | | | | | | | |
| | QLNDSSEEEDEIDGP | 40 | 243 | 8 | EPDRAHYNIVTFCCK | 64 | 10 | 9 |
| DRB1*0401 | SSEEEDEIDGPAGQA | 42 | 235 | 12 | RAHYNIVTFCCKCDS | 61 | 8 | 22 |
| (DR4Dw4) | | | | | | | | |
| | QLNDGP | 43 | 243 | 12 | HYNIVTFCCKCD | 63 | 8 | 14 |
| Peptides of 8, 9, 10 or 15 amino acids from the E7 high-similarity EQLNDSSEEEDEIDGPAGQAE (SEQ ID NO:106) and low-similarity AEPDRAHYNIVTFCCKCDSTL (SEQ ID NO:107) sequences were tested. The viral protein motifs able to bind HLA molecules (see the peptide sequence column) were dissected into 5-mer probes and analyzed for human matches as described in Materials and Methods. The total number of 5-mer matches is reported. The score was calculated by giving the amino acids of a certain | | | | | | | | |
| # peptide a specific value depending on whether they are anchor, auxiliary anchor or preferred residues. Amino acids having a negative effect on the binding ability were evaluated by a negative value (http://www.uni-tuebingen.de/uni/kxi/). Only the first two highest values are reported for each n-mer series, (—), No HLA binding peptide motif found. | | | | | | | | |

### Example 2

The HPV16 E7 oncoprotein sequence was analyzed for 15-mer peptides able to bind to mouse MHC II molecules using the SYFPEITH database of MHC II ligands and peptide motifs. Table 3 reports the ligation strength to class II I-A^{k} and I-E^{k} molecules for 15-mer motifs derived from the entire viral E7 oncoprotein. The analysis of Table 3 shows that a number of E7 15-mer peptides have a value score for MHC II binding potential higher than 10.

TABLE 3

Molecular Mimicry Level and Binding Potential to
MHC II Molecules of 15-mer Peptides from the HPV16
E7 Oncoprotein Sequence.

| | | | SEQ | | Matches |
|---|---|---|---|---|---|
| | Aa | | ID | | to mouse |
| MHC II | position | Peptide Sequence | NO: | Score^{a} | proteome^{b} |
| H2-A^{k} | 18 | ETTDLYCYEQLNDSS | 65 | 18 | 18 |
| | 27 | QLNDSSEEEDEIDGP | 40 | 18 | 282 |
| | 36 | DEIDGPGQAEPDRA | 66 | 18 | 33 |
| | 59 | CKCDSTLRLCVQSTH | 67 | 18 | 23 |
| | 72 | THVDLRTLEDLLMGT | 68 | 18 | 37 |
| | 2 | HGDTPTLHEYMLDLQ^{c} | 69 | 14 | 2 |
| | 26 | EQLNDSSEEEDEIDG | 70 | 14 | 285 |
| | 84 | MGTLGIVCPICSQKP | 71 | 14 | 17 |
| | 11 | YMLDLQPETTDLYCY | 72 | 12 | 19 |
| | 33 | EEEDEIDGPAGQAEP | 73 | 12 | 156 |
| | 45 | AEPDRAHYNIVTFCC | 74 | 12 | 4 |
| | 78 | TLEDLLMGTLGIVCP | 75 | 12 | 43 |
| H2-E^{k} | 25 | YEQLNDSSEEEDEID^{c} | 76 | 20 | 286 |
| | 49 | RAHYNIVTFCCKCDS^{c} | 61 | 20 | 4 |
| | 66 | RLCVQSTHVDIRTLE | 77 | 18 | 53 |
| | 51 | HYNIVTFCCKCDSTL | 63 | 16 | 6 |
| | 73 | HVDIRTLEDLLMGTL | 78 | 16. | 39 |
| | 76 | IRTLEDLLMGTLGIV | 79 | 16 | 52 |
| | 84 | MGTLGIVCPICSQKP | 71 | 16 | 17 |
| | 10 | EYMLDLQPETTDLYC | 80 | 14 | 19 |
| | 19 | TTLDLYCYEQLNDSSE | 81 | 14 | 19 |
| | 35 | EDEIDGPAGQAEPDR | 82 | 14 | 31 |
| | 62 | DSTLRLCVQSTHVDI | 83 | 14 | 66 |
| | 80 | EDLLMGTLGIVCPIC | 84 | 14 | 22 |
| | 22 | LYCYEQLNPSSEEED | 85 | 12 | 178 |
| | 38 | IDGPAGQAEPDRAHY | 86 | 12 | 33 |

^{a.}The score measures thepeptide binding potential. Only values >10 are reported.
^{b.}The BPV16 E7 15-mer peptides able to bind MHC II molecules (see the colunm Peptide sequence) were dissected into 5-mer probes and analyzed for matches to mouse proteome. The total number of 5-mer matches is reported.
^{c.}Selected peptides were chosen for dot immunoassay analysis.

The viral 15-mer peptides predicted to bind the mouse MHC II molecules were analyzed for the level of similarity to mouse proteome sequences. The oncoprotein sequence was dissected into sequential 5-mer motifs offset by one residue, i.e. MHGDT, HGDTP, GDTPT, etc., that were used as sequence probes in computer-assisted similarity analyses. Table 3 reports the total number of matches to mouse proteome for viral 15-mer peptides predicted to bind to MHC II molecules with a ligation strength higher than 10. It can be seen that wide spectrum of similarity levels to mouse proteins (from a maximum of 286 to a minimum of 2 matches) is present among the oncoprotein sequences able to bind to MHC II molecules with a ligation strength >10.

In order to understand the contribution of MHC II binding potential and molecular mimicry in peptide immunodominance, three peptide sequences were devised as possible epitopic determinants in dot immunoassay tests: E7₂₅₋₃₉ YEQLNDSSEEEDEID (SEQ ID NO:76); E7₄₉₋₆₃ RAHYNIVTFCCKCDS (SEQ ID NO:61); E7₂₋₁₆ HGDTPTLHEYMLDLQ (SEQ ID NO:69). As reported in Table 1, the three peptide sequences were representatives, in order, of: i) the highest probability of being presented and high level of similarity to mouse proteins; ii) the highest probability of being presented, and a low level of similarity to mouse proteins; iii) by far the lowest degree of similarity to mouse proteins.

The peptides corresponding to the three peptide sequences were synthetized and used as antigens in dot immunoassay experiments with MAb-ED17, a mouse monoclonal IgG, raised to the full length E7 oncoprotein. Peptide purity was controlled by analytical HPLC, and the molecular mass of purified peptides confirmed by fast atomic bombardment mass spectrometry. Peptides were dissolved in 0.9% NaCl, aliquoted and stored at −20° C.

Nitrocellulose membranes (Nytran 0.2 mm pore size, Schleicher & Schüll) were pretreated for 1 min in 4% BSA (bovine serum albumin)/10 mM Tris-HCl, pH 7.5/150 mM NaCl, followed by 10 min activation with 2.5% glutaraldehyde. Peptides (5 Φg) were spotted on the activated membrane, left to dry for 1 hr at room temperature, and probed in phosphate-buffered saline (PBS) containing 4% BSA, 0.1% (v/v) Tween 20, and the primary antibody (1:500). Primary antibody was mouse anti-HPV16 E7 monoclonal IgG1 raised to amino acids 1-98 representing full length E7 (ED17, cat # sc-6981, Santa Cruz Biotechnology, Inc., Santa Cruz, Calif.). Following a 1 h incubation at room temperature, the membrane was washed three times for 10 mins with PBS containing 4% BSA, 0.1% Tween-20 and incubated with horseradish peroxidase-conjugated affinity-purified sheep anti-mouse IgG for 1 h (1:2500; Santa Cruz Biotechnology). Membrane was washed in PBS (4 times for 5 mins), and immunoblots were developed using the enhanced chemiluminescence detection assay (ECL Western blotting analysis system, Amersham Pharmacia Biotech, Milan, Italy).

Significant binding to MAb-ED17 was observed for the peptide antigen RAHYNIVTFCCKCDS (SEQ ID NO:61) having both the highest binding potential to the MHC II molecules (score=20) and a low degree of similarity to mouse proteoma (number of matches=4). The synthetic peptide HGDTPTLHEYMLDLQ (SEQ ID NO:69) having almost no similarity to mouse protein sequences (number of matches to mouse proteoma=2), but not endowed with the highest MHC II binding potential (score=14), was not recognized by the commercial mAb. Similarly, no binding was observed to the mouse mAb using the 15-mer peptide YEQLNDSSEEEDEID (SEQ ID NO:76) having the highest score for MHC II binding potential (binding potential score=20) and a high level of similarity to mouse proteome (matches to mouse proteoma=286). To confirm the epitope screening results, NMR spectra were obtained that confirmed the high affinity of MAb-ED17 towards the predicted epitopic peptide RAHYNIVTFCCKCDS (SEQ ID NO:61).

The identification of the H2-E^{k} presented HPV 16 E7 epitope was further ananyzed by epitope mapping. Dot immunoassays by using 6-mer peptides offset by one amino acid residue confirmed that the anti-E7 mAb recognized the linear determinant HPV 16 E₅₂₋₆₁ YNIVTFCCKC (SEQ ID NO:108) present in the 15-mer peptide RAHYNIVTFCCKCDS (SEQ ID NO:61), having the highest binding potential to the mouse MHC II molecule, and a low degree of similarity to host proteins.

### Example 3

The HPV16 E7 oncoprotein sequence was further analyzed for 15-mer peptides able to bind to mouse MHC class II I-A^{d} and I-E^{d}. Table 4 reports the peptide sequences and ligation strength for 15-mers having a score for binding potential higher than 14.

TABLE 4

Molecular Mimicry Level and Binding Potential to
MHC II Molecules of 15-mer Peptides from the HPV16
E7 Oncoprotein Sequence.

| | | | SEQ | | Matches |
|---|---|---|---|---|---|
| | Aa | | ID | | to mouse |
| MHC II | position | Peptide Sequence | NO: | Score^{a} | proteome^{b} |
| H2-A^{d} | 84 | MGTLGIVCPICSQKP^{c} | 71 | 22 | 17 |
| | 20 | TDLYCYEQLNPSSEE | 87 | 20 | 29 |
| | 34 | EEDEIDGPGQAEPD | 88 | 20 | 41 |
| | 61 | CDSTLRLCVQSTHVD | 89 | 20 | 67 |
| | 68 | CVQSTHVDIRTLEDL | 90 | 20 | 66 |
| | 39 | DGPAGQAEPDRAHYN | 91 | 19 | 30 |
| | 2 | HGDTPTLHEYMLDLQ^{c} | 69 | 18 | 2 |
| | 7 | TLHEYMLDLQPETTD | 92 | 17 | 16 |
| | 76 | IRTLEDLLMGTLGIV | 79 | 16 | 52 |
| | 59 | CKCDSTLRLCVQSTH | 67 | 15 | 23 |
| | 32 | SEEEDEIDGPAGQAE^{c} | 39 | 14 | 262 |
| | 60 | KCDSTLRLCVQSTHV | 93 | 14 | 68 |
| | 63 | STLRLCVQSTHVDIR | 94 | 14 | 62 |
| | 77 | RTLEDLLMGTLGIVC | 95 | 14 | 48 |
| H2-E^{d} | 49 | RAHYNIVTFCCKCDS^{c} | 61 | 18 | 4 |
| | 54 | IVTFCCKCDSTLRLC | 96 | 16 | 20 |
| | 66 | RLCVQSTHVDIRTLE | 77 | 16 | 53 |
| | 71 | STHVDIRTLEDLLMG | 97 | 14 | 35 |

^{a.}The score measures the peptide binding potential. Only values ≧14 are reported.
^{b.}The BPV16 E7 15-mer peptides able to bind MHC II molecules (see the colunm Peptide sequence) were dissected into 5-mer probes and analyzed for matches to mouse proteome. The total number of 5-mer matches is reported.
^{c.}Selected peptides were chosen for dot immunoassay analysis.

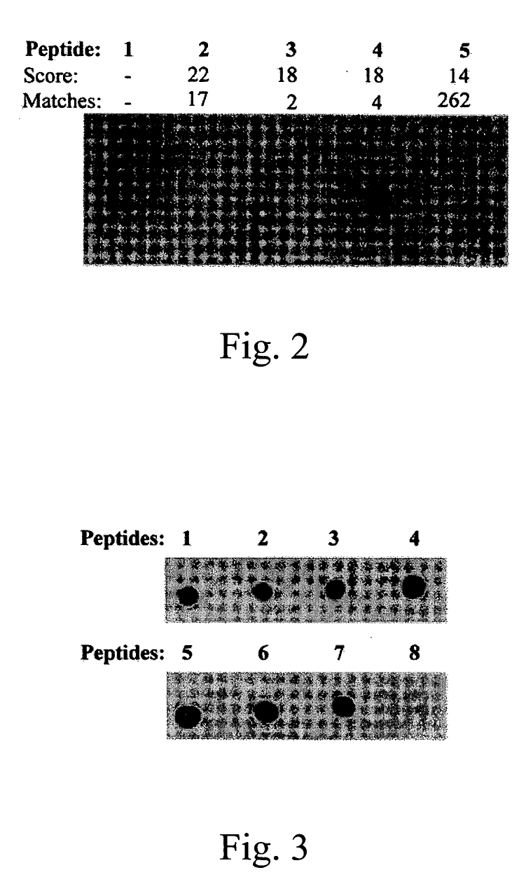

Four peptides were analyzed for epitopic determinants in dot immunoassay tests: E7₂₅₋₃₉ YEQLNDSSEEEDEID (control; SEQ ID NO:76); E7₈₄₋₉₈ MGTLGIVCPICSQKP (SEQ ID NO:71); E7₂₋₁₆ HGDTPTLHEYMLDLQ (SEQ ID No:69); E7₄₉₋₆₃ RAHYNIVTFCCKCDS (SEQ ID NO:61); and E7₃₂₋₄₆ SEEEDEIDGPAGQAE (SEQ ID NO:39). As reported in FIG. 2, E7₈₄₋₉₈ MGTLGIVCPICSQKP (SEQ ID NO:71), having the highest ligation strength for H2-Ad, but also a high level of similarity to mouse proteome (FIG. 2, peptide 2), was not recognized by the commercial anti-E7 mAb. No immune reaction was observed with mAb ED17 by using the 15-mer peptide E7₂₋₁₆ HGDTPTLHEYMLDLQ (SEQ ID NO:69) having almost zero similarity to the mouse protein sequences and endowed with a moderate MHC II binding potential (FIG. 1, peptide 3). As expected, high similarity peptide E7₃₂₋₄₆ SEEEDEIDGPAGQAE (SEQ ID NO:39) was not reactive (FIG. 1, peptide 5). A significant signal was observed using the peptide E7₄₉₋₆₃ RAHYNIVTFCCKCDS (SEQ ID NO:61) having both the highest binding potential to H2-E^{d} molecules and a low degree of similarity to the mouse proteome.

E7₄₉₋₆₃ RAHYNIVTFCCKCDS (SEQ ID NO:61) was further analyzed by epitope mapping. As illustrated in FIG. 2, dot immunoassays by using 6-mer peptides offset by one amino acid residue confirmed that mAb ED17 recognized the linear determinant HPV16 E7₅₀₋₆₁ AHYNIVTFCCKC present in the 15-mer peptide.

### Example 4

In a similar experiment, using a model breast/prostate cancer-associated HER-2/neu antigen, polyclonal and monoclonal responses were analyzed. The HER-2/neu oncoprotein was scanned for similarity to the mouse and human proteomes. The extracellular domain was divided into 5-mer sequences offset by one amino acid. As described above for HPV E7, 10 amino acid peptides of differing sequence similarities were synthesized and tested in immunoassays. A commercial monoclonal antibody was found to bind to a peptide in a low similarity group having only three matches with the mouse proteome. The synthetic peptides were also tested with polyclonal sera from breast/prostate cancer patients. It was found that poorly shared motifs were preferentially recognized by the polyclonal antibody populations.

What is claimed is:

1. A method of identifying an immunodominant epitope of an antigen comprising:
examining amino acid sequences within the antigen for binding affinity to an MHC molecule;
examining amino acid sequences within the antigen to determine sequence similarity to the host proteome; and
selecting an amino acid sequence within the antigen having high MHC binding affinity and low sequence similarity to the host proteome.

2. The method of claim 1, wherein the MHC molecule is a class I MHC molecule.

3. The method of claim 1, wherein the MHC molecule is a class II MHC molecule.

4. The method of claim 1, wherein binding affinity is predicted by comparing amino acid sequences within the antigen to a consensus MHC binding sequence.

5. The method of claim 1, wherein sequence similarity is examined by comparing overlapping amino acid sequences within the antigen to the host proteome.

6. The method of claim 5, wherein the overlapping amino acid sequences are 4 to 10 amino acids in length.

7. The method of claim 5, wherein the overlapping amino acid sequences are 5, 6, or 7 amino acids in length.

8. The method of claim 5, wherein the short overlapping amino acid sequences are offset by 5 amino acids.

9. The method of claim 5, wherein the short overlapping amino acid sequences are offset by 1 or 2 amino acids.

10. A method of producing a polypeptide useful for eliciting an immune response against an antigen in a host comprising;
(a) analyzing amino acid sequences within the antigen for binding affinity to an MHC molecule;
(b) examining amino acid sequences within the antigen to determine sequence similarity to the host proteome;
(c) selecting an amino acid sequence having high MHC binding affinity and low sequence similarity; and
(d) producing a polypeptide comprising the selected amino acid sequence.

11. The method of claim 10, wherein binding affinity is predicted by comparing amino acid sequences within the antigen to a consensus MHC binding sequence.

12. The method of claim 10, wherein sequence similarity is examined by comparing short overlapping amino acid sequences within the antigen to the host proteome.

13. A method of eliciting a therapeutic immune response to an antigen comprising administering to a subject an effective amount a polypeptide comprising an amino acid selected by:
(a) analyzing amino acid sequences within the antigen for binding affinity to an MHC molecule;
(b) examining amino acid sequences within the antigen to determine sequence similarity to the host proteome; and
(c) selecting an amino acid sequence having high MHC binding affinity and low sequence similarity.

14. The method of claim 13, wherein the MHC moleucle is a class II MHC molecule and the therapeutic response is a humoral response.

15. The method of claim 13, wherein the selected amino acid sequence comprises a B cell epitope.

16. The method of claim 13, wherein the polypeptide further comprises a B cell epitope linked to the selected amino acid sequence.

17. The method of claim 13, wherein the MHC molecule is a class I MHC molecule and the therapeutic response is a cytotoxic cellular response.

18. The method of claim 13, wherein the antigen is a tumor antigen.

19. The method of claim 13 wherein the antigen is from an infectious agent.
